(19) United States
(12) Patent Application Publication
(10) Pub. No.: US 2016/0092613 A1
(43) Pub. Date: Mar. 31, 2016

(54) SYSTEMS AND METHODS FOR PERFORMING A REDUCED ORDER APPROXIMATION TO MODEL AN ABSORBENT ARTICLE

(71) Applicant: The Procter & Gamble Company, Cincinnati, OH (US)

(72) Inventors: James Kenneth COMER, JR., West Chester, OH (US); Ward William OSTENDORF, West Chester, OH (US); Peter Randall SCHUNK, Albuquerque, NM (US)

(21) Appl. No.: 14/497,900

(22) Filed: Sep. 26, 2014

(51) Int. Cl. G06F 17/50; A61F 13/15

(52) U.S. Cl. CPC G06F 17/5009; A61F 13/15

(57) ABSTRACT

Included are embodiments for performing a reduced order approximation to model an absorbent article. Some embodiments include assigning at least one material property of the absorbent article, wherein the absorbent article includes a plurality of absorbent plies, assigning at least one initial condition and at least one boundary condition associated with a fluid to be virtually introduced to the absorbent article, performing a reduced order approximation to create a simulation of the absorbent article, based on the at least one material property and the at least one initial condition, and providing the simulation for display.

### FIELD OF THE INVENTION

The present application relates generally to systems and methods for performing a reduced order approximation to model an absorbent article and specifically to systems and methods that determine absorbency characteristics of an absorbent article that is introduced to a fluid.

### BACKGROUND OF THE INVENTION

When designing absorbent articles, such as paper towels, toilet paper, diapers, feminine products, household cleaning products, and the like, there are many design characteristics that are conceived and implemented. As such, many current solutions involve the design of an absorbent article, the physical manufacture of that absorbent article, and the testing of the absorbent article in varying conditions. While such a workflow may provide a desired end product, the financial and time costs of designing, manufacturing, testing, and redesigning the absorbent article are often difficult for the designer to overcome.

### SUMMARY OF THE INVENTION

Included are embodiments of a system for modeling an absorbent article. Accordingly, these embodiments of the system include a memory component that stores logic that, when executed by a processor, causes the system to assign at least one material property of the absorbent article, where the absorbent article includes a plurality of absorbent plies, and wherein the absorbent article includes a plurality of absorbent regions, assign at least one initial condition and at least one boundary condition associated with a fluid to be virtually introduced to the absorbent article, perform a reduced order approximation to create a simulation of the plurality of absorbent regions, based on the at least one material property and the at least one initial condition, and provide the simulation for display.

Also included are embodiments of a method for modeling an absorbent article. Some embodiments of the method include assigning at least one material property of the absorbent article, wherein the absorbent article includes a plurality of absorbent plies, assigning at least one initial condition and at least one boundary condition associated with a fluid to be virtually introduced to the absorbent article, performing a reduced order approximation to create a simulation of the absorbent article, based on the at least one material property and the at least one initial condition, and providing the simulation for display.

Also included are embodiments of a non-transitory computer-readable medium. Some embodiments of the non-transitory computer-readable medium include logic that, when executed by a computing device, causes the computing device to assign at least one material property of the absorbent article, wherein the absorbent article includes a plurality of absorbent plies, wherein the absorbent article includes a plurality of absorbent regions, assign at least one initial condition and at least one boundary condition associated with a fluid to be virtually introduced to the absorbent article, perform a shell formulation to create a simulation of the plurality of absorbent regions, based on the at least one material property and the at least one initial condition, and provide the simulation for display.

### BRIEF DESCRIPTION OF THE DRAWINGS

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate various embodiments described herein, and together with the description serve to explain the principles and operations of the claimed subject matter.

FIG. 1 depicts a user computing device for modeling an absorbent article, according to embodiments disclosed herein;

FIGS. 2A-2D depict a plurality of conditions for a source of fluid to interact with an absorbent article, according to embodiments disclosed herein;

FIGS. 3A, 3B depict interaction of a fluid with a multi-ply absorbent article, according to embodiments disclosed herein;

FIG. 4 depicts a user interface for modeling the absorbent article, according to embodiments disclosed herein;

FIG. 5 depicts a model of an absorbent article for the creation of a plurality of cells in the absorbent article, according to embodiments disclosed herein;

FIG. 6 depicts a plurality of plies of an absorbent article, as may be modeled by the computing device, according to embodiments disclosed herein;

FIGS. 7A-7C depict a representative mapping of a multi-ply absorbent article for determining an interaction among plies, according to embodiments disclosed herein;

FIG. 8 depicts a simulation of interaction between a fluid and the absorbent article, according to embodiments disclosed herein;

FIG. 9 depicts a flowchart for determining whether a simulated absorbent article meets predetermined performance characteristics, according to embodiments disclosed herein;

FIG. 10 depicts a flowchart for determining whether the simulated material meets predetermined performance characteristics, according to embodiments disclosed herein;

### DETAILED DESCRIPTION OF THE INVENTION

Embodiments disclosed herein include systems and methods for modeling an absorbent article. Specifically, embodiments described herein include porous media modeling where capillary wicking may play a substantial role. These embodiments may be configured to simulate the coupling of thin porous media with adjacent free surface flow domains, as well as simulate flow on top of a thin absorbent article, between a plurality of absorbent plies of similar or different properties and between the absorbent article and impermeable or permeable boundaries. Some embodiments are configured to model and/or simulate a plurality of different configurations, including single thin porous layer, multiple thin porous layers, thin free surface regions, coupling of free surface domains with thin porous layers, and coupling the thin porous media models with traditional three dimensional porous media models. Embodiments may also be configured to couple a localized “sink” term with the thin porous shell formulation, which allows the capture of the behavior of absorbent materials as well as super absorbent materials.

Similarly, some embodiments are configured to directly map material height, thickness and/or related properties to the computational simulation domain for three dimensional individual and multi-ply structures based on two-dimensional sketches, patterns, and/or pictures. Similarly, some embodiments may have the ability of free-liquid to leave a porous media and move into the open portion of the domain (e.g., the inter-ply gap). This is a numerical construct which may be utilized to mimic nucleation of liquid between porous domains and free-liquid domains. Embodiments may also be configured to simulate movement of absorbent articles (e.g., wiping) by enabling the simulation of relative motion between the absorbent article and the boundary embodiments to provide for the study of how residual fluid (such as a Newtonian and/or non-Newtonian fluid) and dynamic motion are related. Simulation of relative motion between the absorbent article and a surface provides a more realistic in-use representation for many consumer relevant tasks than static only simulations.

Embodiments may also be configured to study the competition between gravitational and/or inertial effects on the free fluid flow and the absorbent structure, as well as the impact of thin deforming structures on liquid transport and/or absorbent characteristics of the absorbent article. This allows for the simulation of flow on top of the absorbent article (such as a capillary based structure), between multiple absorbent plies of similar or different properties, and between the absorbent article and impermeable or permeable boundaries. Current embodiments may also use a “sink” term to capture the behavior of cellulose materials, superabsorbent materials, starch materials, etc. It should also be noted that, depending on the particular embodiment, absorbency may be determined and/or modeled between plies of the material, within a porous matrix of a particular ply, and/or within the matrix material itself.

While the above discussion deals with the ability to use a shell formulation to simulate flow in thin porous structures in a standalone mode. Some embodiments may be configured to couple the thin shell elements with traditional continuum domain elements. Such functionality may be utilized for thick material absorbent products like those in diapers, feminine products, adult incontinence products, etc. An example might include structures that have one or more thin layers which are adjacent to more traditional “thick” porous media or a situation where you have a thin porous structure next to a large free domain which is not described by the thin lubrication approximation. Embodiments may additionally utilize separate shell elements for various layers.

Embodiments may also be configured to provide for the study of deforming structures. The basic capability that is built into this approach enables the deformation of the shells due to internal and/or external loads. Embodiments may also be configured for developing a thin-shell porous flow model, together with a coupling to a lubrication region which represents the liquid to be soaked up by the thin structure, whether as a single ply absorbent article or as a multi-ply absorbent article.

Accordingly, embodiments described herein may be configured to approximate a thin porous structure and/or a structure comprising a plurality of thin structures (layers) with a two-dimensional numerical simulation. Some embodiments utilize a three dimensional shell formulation or other reduced order approximation. As will be understood, the reduced-order approximation may be configured to convert an n dimension initial representation into an n-1 dimension simulation (where n is greater than 1). Similarly, embodiments herein may be configured to perform this simulation for multiple layers and a free liquid domain. To this end, the following publications are hereby incorporated by reference in their entireties: Roberts, S. A.; Noble, D. R.; Benner, E. M. & Schunk, P. R. (2013), “Multiphase hydrodynamic lubrication flow using a three dimensional shell finite element model,” Computers & Fluids. Volume 87, 25 Oct. 2013; and Roberts, S. A. & Schunk, P. R. (2013), “Porous shell model development for thin, structured materials” Computers & Fluids, submitted, and Roberts, S. A.; Schunk, P. R. (2014), “A reduced-order model for porous flow through thin, structured materials,” International Journal of Multiphase Flow. Volume 67, 25 Aug. 2014.

It should also be understood that, while some embodiments described herein are configured to model and/or optimize the absorbency of an absorbent article of a fluid from a surface, these are merely examples. Some embodiments may be configured to model and/or optimize an absorbent article that stores a fluid to be removed from the absorbent article onto a surface. As such, similar computations may be utilized for each scenario, as described in more detail below. Regardless, embodiments may be configured to model the absorbent article and/or the surface between which the fluid transfers.

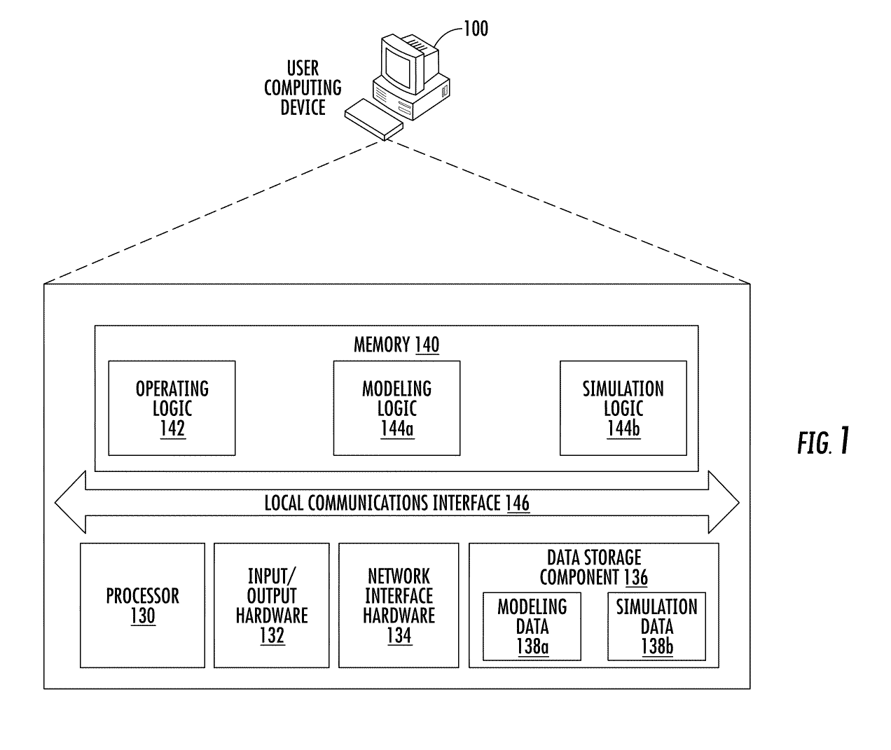

Referring now to the drawings, FIG. 1 depicts a user computing device 100 for modeling an absorbent article, according to embodiments disclosed herein. As illustrated, the user computing device 100 may include a processor 130, input/output hardware 132, network interface hardware 134, a data storage component 136 (which stores modeling data 138a, and simulation data 138b), and the memory component 140. The memory component 140 may be configured as volatile and/or nonvolatile memory and as such, may include random access memory (including SRAM, DRAM, and/or other types of RAM), flash memory, secure digital (SD) memory, registers, compact discs (CD), digital versatile discs (DVD), and/or other types of non-transitory computer-readable mediums. Depending on the particular embodiment, these non-transitory computer-readable mediums may reside within the user computing device 100 and/or external to the user computing device 100.

The memory component 140 may store operating logic 142, modeling logic 144a, and simulation logic 144b. The modeling logic 144a and the simulation logic 144b may each include a plurality of different pieces of logic, each of which may be embodied as a computer program, firmware, and/or hardware, as an example. A local interface 146 is also included in FIG. 1 and may be implemented as a bus or other communication interface to facilitate communication among the components of the user computing device 100.

The processor 130 may include any processing component operable to receive and execute instructions (such as from a data storage component 136 and/or the memory component 140). Similarly, the network interface hardware 134 may include and/or be configured for communicating with any wired or wireless networking hardware, including an antenna, a modem, a LAN port, wireless fidelity (Wi-Fi) card, WiMax card, mobile communications hardware, and/or other hardware for communicating with other networks and/or devices. From this connection, communication may be facilitated between the user computing device 100 and other computing devices across a network, such as a local area network and/or the internet.

The operating logic 142 may include an operating system and/or other software for managing components of the user computing device 100. Similarly, as discussed above, the modeling logic 144a may reside in the memory component 140 and may be configured to cause the processor 130 to model an absorbent article. Similarly, the simulation logic 144b may be utilized to analyze data from the user for creating the user interfaces and to simulate introduction of a fluid with the absorbent article.

It should be understood that while the components in FIG. 1 are illustrated as residing within the user computing device 100, this is merely an example. In some embodiments, one or more of the components may reside external to the user computing device 100. It should also be understood that, while the user computing device 100 is illustrated as a single device, this is also merely an example. In some embodiments, the modeling logic 144a and the simulation logic 144b may reside on different computing devices. As an example, one or more of the functionalities and/or components described herein may be provided by the user computing device 100 or other computing device.

Additionally, while the user computing device 100 is illustrated with the modeling logic 144a and the simulation logic 144b as separate logical components, this is also an example. In some embodiments, a single piece of logic may cause the user computing device 100 to provide the described functionality.

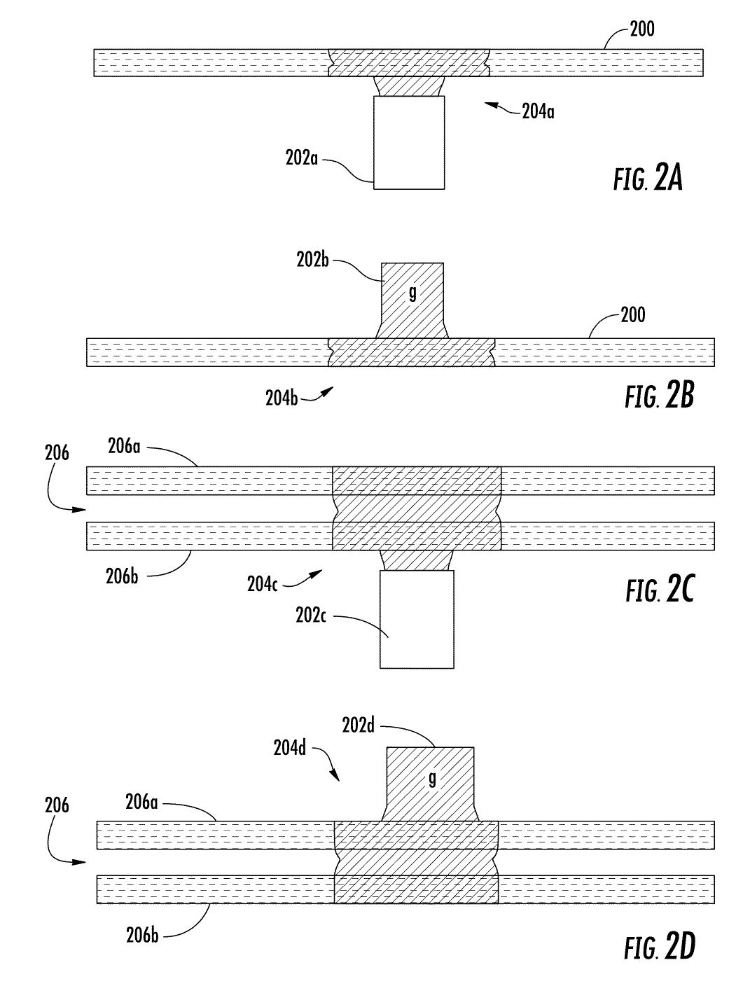

FIGS. 2A-2D depict a plurality of conditions for a fluid 204 to interact with an absorbent article 200, 206, according to embodiments disclosed herein. Specifically, these embodiments may be configured to simulate contact of a fluid 204 with an absorbent article in a plurality of different ways. This interaction may come from a finite source or an infinite source and may apply to a thin single-ply material or a multi-ply absorbent article (such as a thin double-ply material, thin triple-ply material, etc.). Accordingly, FIG. 2A illustrates a single-ply absorbent article with an infinite source 202a that provides a fluid 204a that interacts with the single-ply absorbent article 200. The infinite source 202a may provide an infinite amount of fluid to be introduced to the single-ply absorbent article 200. Example FIG. 2B includes fixed liquid loading with redistribution. FIG. 2B illustrates the single-ply absorbent article 200 and a finite source 202b for providing a finite amount of a fluid. FIG. 2C illustrates an infinite source 202c that is providing an infinite amount of a fluid 204c to a multi-ply absorbent article 206 with plies 206a and 206b. FIG. 2D illustrates a finite source 202d that provides a finite amount of a fluid 204d to the multi-ply absorbent article 206, with plies 206a and 206b.

It should be understood that while the embodiments of FIGS. 2A-2D illustrated single-ply and double-ply absorbent articles, these are merely examples. Embodiments described herein may be utilized to model and/or simulate multi-ply absorbent articles, such as three-ply (triple-ply) or more. Additionally, while some of the examples described herein refer to thin absorbent articles, embodiments may be configured to model and/or simulate thick absorbent articles, such as those in the feminine care industry, the cleaning industry, the diaper industry, etc. Other scenarios, such as those with a fluid on a surface for both single-ply and multi-ply absorbent articles may also be included for modeling and simulation as described herein.

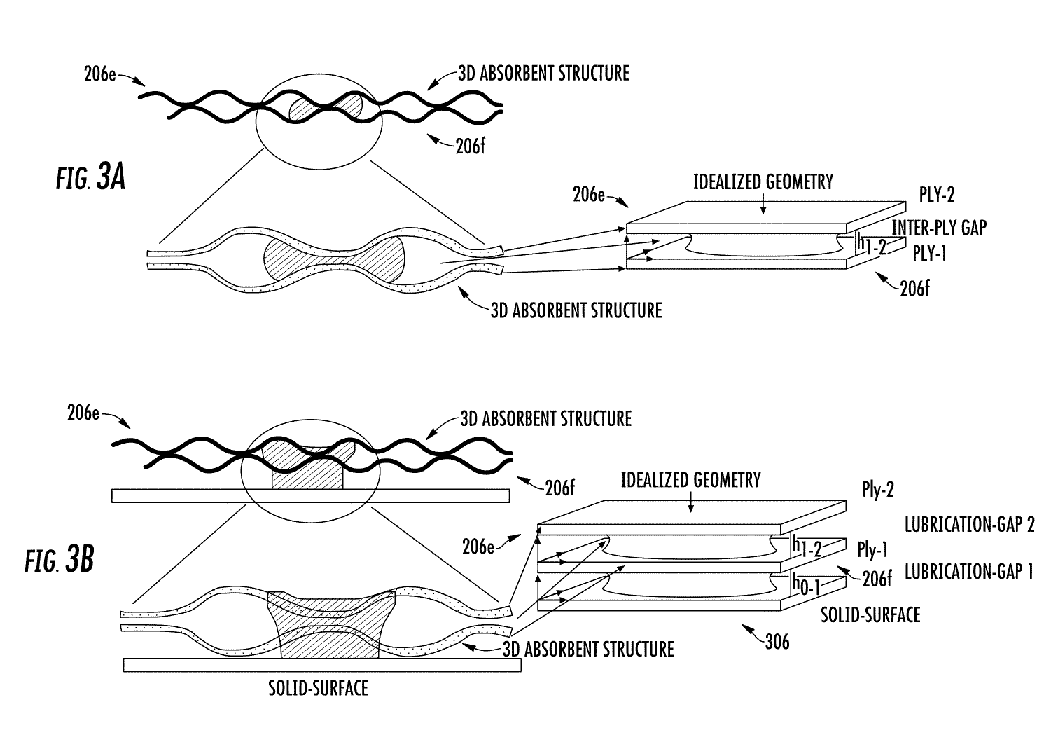

FIGS. 3A, 3B depict interaction of a fluid with a multi-ply absorbent article 206, according to embodiments disclosed herein. Specifically in FIG. 3A, an absorbent article 206 may be designed as a thin article with a non-flat profile. Accordingly, when a multi-ply absorbent article is constructed, one or a plurality of absorbent regions and/or topographies may be created. As an example, the regions may be referred by “pillows” and/or “knuckles,” which may represent areas of positive or negative change with respect to a predetermined plane axis. Each of the plies 206e, 206f may have high points and low points. Accordingly, when a predetermined type and amount of fluid interacts with the absorbent article, the plurality of regions will provide a wicking mechanism to absorb and distribute the fluid. Depending on the particular embodiment, the three dimensional material may have the same properties throughout or each region may have different properties, which may be defined specifically.

Also depicted in FIG. 3A is a graphical representation of the idealized geometry of the absorbent article 206. As discussed above, embodiments described with regard to FIG. 3A may be configured to simulate interaction of the fluid between the first ply 206f and the second ply 206e. Specifically, the distance between the two plies 206e, 206f may be depicted has “h₁₋₂,” which may be referred to as an inter-ply gap.

FIG. 3B also shows an idealized geometry on the right with the thin sheet regions corresponding to each ply. Because of the generalizable shell-element formulation, which may be utilized to create the simulation, there is no reason why the shell element layers could not be made to conform to the corrugated geometry as in the real structure on the left portion of FIG. 3A. This simplified model takes each ply as substantially flat from a microscopic level (e.g., at the cell level), while the structure may be curved or otherwise not flat from a macroscopic level. The model is one of the absorption of two initially bridging drops: the first between a surface 306 (e.g. table top) and the first ply 206f, and the second between the two plies 206f, 206e. The drops do not need to be aligned with one another. In the simulation, the plies 206e, 206f are initially dry or specified to have a very low saturation, at zero time.

Similarly, FIG. 3B depicts a similar idealized geometry with a solid surface being included. Specifically, FIG. 3B depicts the two plies 206e, 206f an in FIG. 3A, as well as the interaction of the fluid between the first ply 206f and a surface 306, such as a table top on which the fluid resides. Accordingly, the simulation may include lubrication gap 1 between the surface 306 and the first ply 206f (“h₀₋₁”) and lubrication gap 2 between the first ply 206f and the second ply 206e (“h₁₋₂”). In such a scenario, the surface 306 may or may not have absorbent qualities and thus may be simulated accordingly.

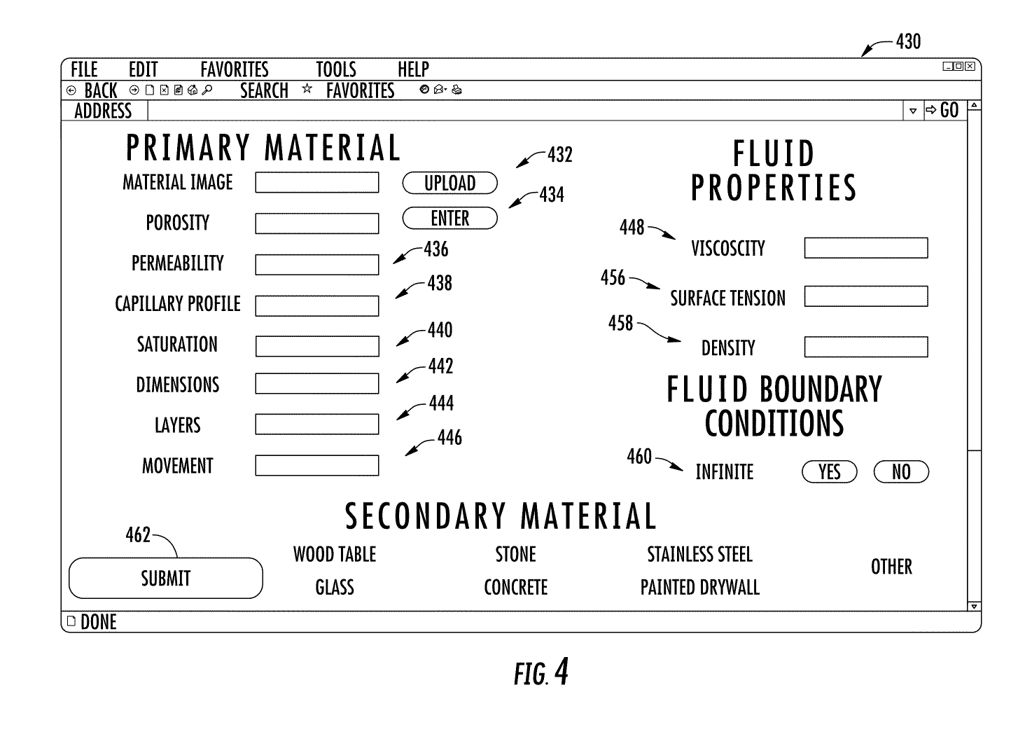

FIG. 4 depicts a user interface 430 for modeling the absorbent article, according to embodiments disclosed herein. As illustrated, the user interface 430 may include a plurality of options for assigning at least one material property of the absorbent article and at least one initial condition associated with a fluid. With this information, the fluid may be virtually introduced with a model of the absorbent article and absorption may be simulated.

Accordingly, the user interface 430 includes a plurality of material property options (and other options), such as a material image option 432, a porosity option 434, a permeability option 436, a capillary profile option 438, a saturation option 440, a dimensions option 442, a layers option 444, and a movement option 446. The material image option 432 may be configured for receiving a two dimensional image (such as .jpg, .gif, etc.) of the absorbent article. The image may be taken from an overhead perspective to illustrate the different regions and other geometric topology of the absorbent article (one or more plies of the absorbent article). Once received, the image may be processed by the user computing device 100 to create a pattern overlaid mesh field. As an example, this may be created using a Gauss-point interpolation, a least squares projection, and/or others. With this image processing there is no need to grid the pattern into the mesh. This can be generalized to other field parameters and properties such as pores height, pillar height, thermo-physical properties, etc.

Similarly, some embodiments may be configured for a user to directly input information about the absorbent article. This may be in addition to uploading the image of the absorbent article or instead of uploading the image. Additionally, porosity of the absorbent article may be entered with the porosity option 434. The porosity may be entered for each individual ply for a multi-ply absorbent article, for each region of the absorbent article, and/or for the absorbent article as a whole. The permeability may be entered, as well as capillary profile, saturation (such as an initial saturation), dimensions, layers, and movement of the absorbent article in the respective options 436-446.

At least one fluid property and/or at least one boundary condition may be entered via options 448-460. The fluid properties refer to conditions of the fluid and other characteristics of the environment that are separate from the absorbent article. The boundary conditions are related to whether the fluid is provided from a finite source or an infinite source and from where the fluid is originating. Accordingly, the user interface 430 includes a viscosity option 448, a surface tension option 456, and a density option 458 and an infinite or finite boundary condition option 460. The viscosity option 448 may allow the user to enter viscosity model parameters for the liquid. The other properties may also be entered for the fluid, depending on the embodiment.

Also included in the user interface 430 are options to select properties of a secondary material, such as a surface on which the fluid will reside. Depending on the particular embodiment, the user may select the type of material as illustrated in FIG. 4 and/or the user may enter individual properties similar to input for the primary material described above.

It should be understood that some embodiments may be configured for a user to select previously stored characteristics for the primary material, the fluid, and/or the secondary material. Additional features of these and/or other characteristics may also be provided. As an example, while movement is an option provided in FIG. 4, greater details (such as direction speed, path, pressure, etc. may be entered by the user to fully describe the details of this aspect of the simulation. Similarly, shell thickness, cross-shell permeability (as used with shell analysis, which may be used for the modeling and/or simulation), and other features of the calculation may also be provided by the user via the user interface 430, user routines, and/or automatically included in the calculation by the user computing device 100. Similarly, the option 460 is also an example, as some embodiments may determine whether the source is a finite or infinite source via other mechanisms, such as uploads, or other user designations.

With this information received from the user, the user computing device 100 may create a model of the absorbent material, fluid, and secondary material. With this modeling, a simulation may be executed to determine the performance characteristics of the absorbent material with the entered conditions.

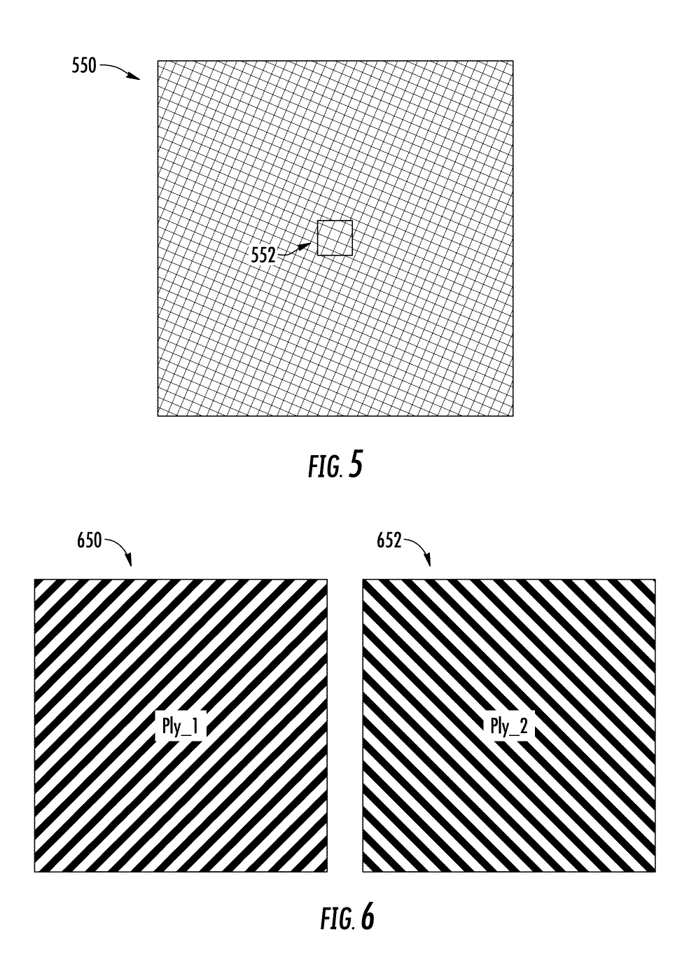

FIG. 5 depicts a model of an absorbent article 550 for the creation of a plurality of mesh-based fields (cells) in the absorbent article, according to embodiments disclosed herein. Specifically, upon receiving the initial conditions and the material properties of the absorbent article as described with regard to FIG. 4, the user computing device 100 may create a three dimensional simulation of the absorbent article. The three dimensional simulation may be divided up into cells. Referring specifically to FIG. 5, the absorbent article 550 is depicted, with a cell 552 being divided. With the cell 552 being divided, the user computing device 100 may perform a simulation with a plurality of the cells to determine the performance characteristics of the absorbent article as a whole.

As an example, the simulation may include applying a predetermined volume (finite or infinite) of the fluid to a predetermined cell of the absorbent article 550. Due to the geographic topography of the absorbent article 550 (e.g., placement of the pillows and knuckles), each cell may have different performance characteristics. Accordingly, based on the wicking, absorbency, and/or other performance characteristics of a first cell, surrounding cells will then be introduced to the fluid. These cells will have individual performance characteristics and will thus react accordingly when the fluid is introduced. Based on this chain reaction of the fluid interaction with each of the individual cells, the performance characteristics of the absorbent article as whole may be simulated.

Accordingly, in some embodiments, mesh-based fields may be created, such as via use of the depiction of the absorbent article 550 in FIG. 5. For these, saturation curves, permeabilities, and gap height may be created with images received via the user interface 430 from FIG. 4.

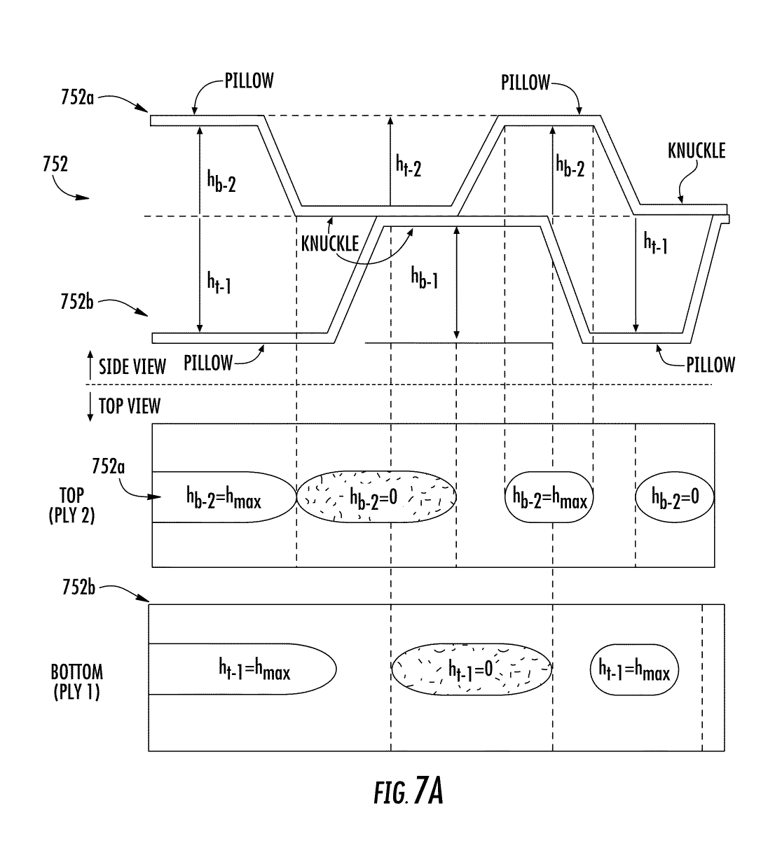

FIG. 6 depicts a plurality of plies 650, 652 of an absorbent article, as may be modeled by the user computing device 100, according to embodiments disclosed herein. As illustrated, the porous properties, such as sheet thickness and the lubrication height are mapped according to a cross-hatched pattern in the first ply 650 and the second ply 652. The black regions correspond to a maximum value of permeability, sheet thickness, cross-sheet permeability, and/or advancing saturation function. The white regions may be taken as the minimums of these functions. The pattern may then be processed to determine the performance capabilities of the absorbent article.

With the information derived from the modeled plies 650, 652, as well as the division of cells described with regard to FIG. 5, the absorbent article may be modeled. With the modeling and initial conditions, one or more simulations may be created and executed to determine the performance characteristics of the modeled absorbent article.

It should be understood that while the depiction in FIG. 6 illustrates a uniform pattern on the material, with two different types of regions, this is merely an example. Some embodiments may be configured to simulate a plurality of different regions and region types, based on differing topologies of each of the plies in the materials. As an example, if a two-ply material includes two types of pillows and two types of knuckles, the material may include at least four different types of regions, each with potentially different performance characteristics.

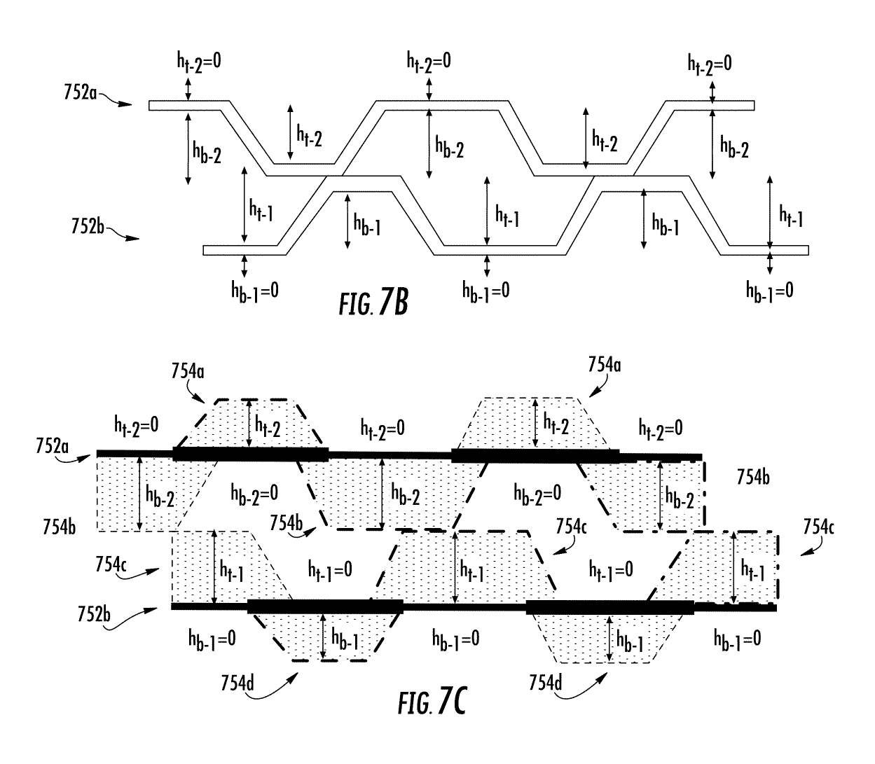

FIGS. 7A-7C depict a mapping of a multi-ply absorbent article 752 for determining an interaction among plies 752a, 752b, according to embodiments disclosed herein. As illustrated in FIG. 7A, a profile of the multi-ply absorbent article 752 may be provided, illustrating the independent plies 752a, 752b. A top view may also be provided to illustrate the area of the absorbent article that corresponds with the different regions and their alignment with each of the plies 752a, 752b. Specifically, a first section of the second ply 752a and a first portion of the first ply 752b align with pillows on each ply. Thus, in this section hₜ₋₁ and h_{b-2} equals hₘₐₓ. In the sections where a knuckle is present, hₜ₋₁ and h_{b-2} equals 0. This analysis of the plies 752a, 752b of the multi-ply absorbent article 752 provides a model for determining the performance characteristics of the multi-ply absorbent article 752.

Accordingly, a computational shell representation of the three dimensional model may be created to identify the performance characteristics of a particular cell of the absorbent article. As an example, where two pillows align, the cell may have first performance characteristics. Where two knuckles align, second performance characteristics may result. Where a knuckle and a pillow align, third performance characteristics may be realized. These characteristics may also vary, based on the material composition, initial conditions, boundary conditions and/or other factors. It should be realized, that depending on the particular embodiment, any of a plurality of different alignments between ply regions may be present.

It should be understood that while a profile depiction and/or analysis may also be provided between a ply and the surface. Generally speaking, the surface may be substantially planar and non-absorbent (such as a tile floor), in which case, the analysis is a comparison of the ply 752b with a baseline that represents the floor. However, some embodiments may include a non-planar and/or absorbent surface, which may add additional complexities to this analysis.

Similarly, FIG. 7B depicts a double ply embodiment of a microscopically flat porous media. Specifically, the second ply 752a may have a plurality of regions, with differing heights. The height may be represented as the free domain below the ply (“h_{b}”) and the free domain above the ply (“hₜ”). On the far left of FIG. 7B, the second ply 752a may have a free domain above the ply as hₜ₋₂=0, since that ply is at a maximum height. At that point, h_{b-2} may have a maximum value, since, the height between the second ply 752a and the ply line would be at a maximum. Similarly, at the same point the hₜ₋₁ has a value that is also at a maximum because the first ply 752b is below the ply line. Similar determinations and calculations may be made for a plurality of regions on the material.

FIG. 7C depicts an implementation, of the free fluid height. Specifically, utilizing the same variable convention, the height of the fluid may be represented in the spaces created in FIG. 7B. Accordingly, the fluid may be represented for the one or more different regions in the material. The inter-ply height between the first ply 752b and the second ply 752a may be referenced as h₁₋₂=(hₜ₋₁+h_{b-2}).

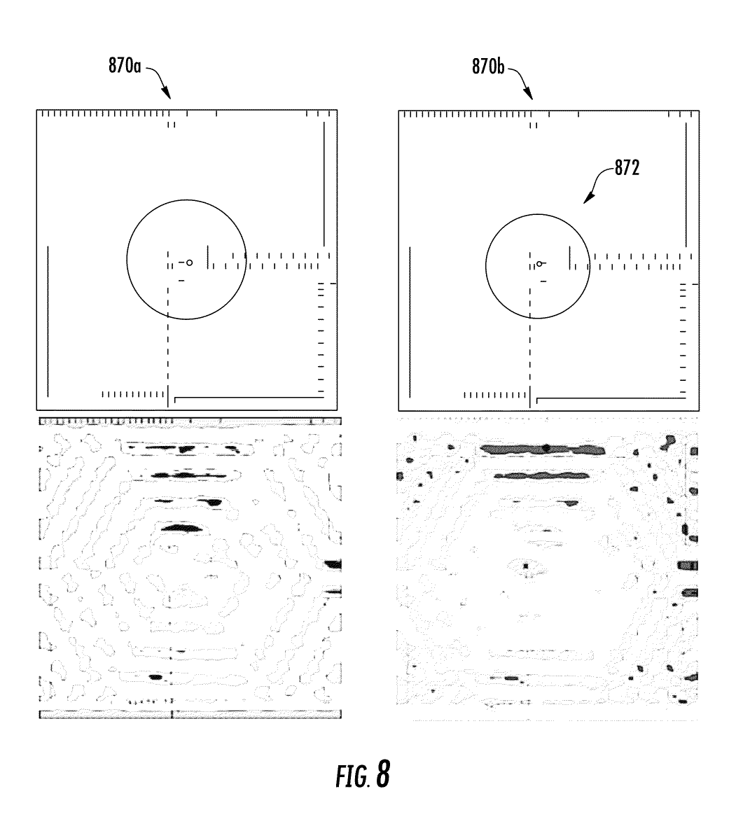

FIG. 8 depicts a simulation 870a, 870b of interaction between a fluid and the absorbent article, according to embodiments disclosed herein. As illustrated, an initial drop configuration 872 and early saturation performance characteristics may be graphically depicted and provided to the user. Based the information provided by the user (such as in FIG. 3), the material topography, as well as the performance characteristics that result from those areas of a plurality of the cells, the performance characteristics may be determined and provided. As will be understood, different colored areas may represent different saturation, absorbency, and/or other performance characteristics. Additionally, the user computing device 100 may be configured to determine whether any portion of the absorbent article meets predetermined criteria for a performance characteristic. If the absorbent article meets the predetermined criteria, the absorbent article may be acceptable for this use. If a portion of the absorbent article does not meet the predetermined criteria, the user computing device 100 may identify the deficiency. In some embodiments, the user computing device 100 may additionally suggest design changes to the absorbent article to optimize performance criteria.

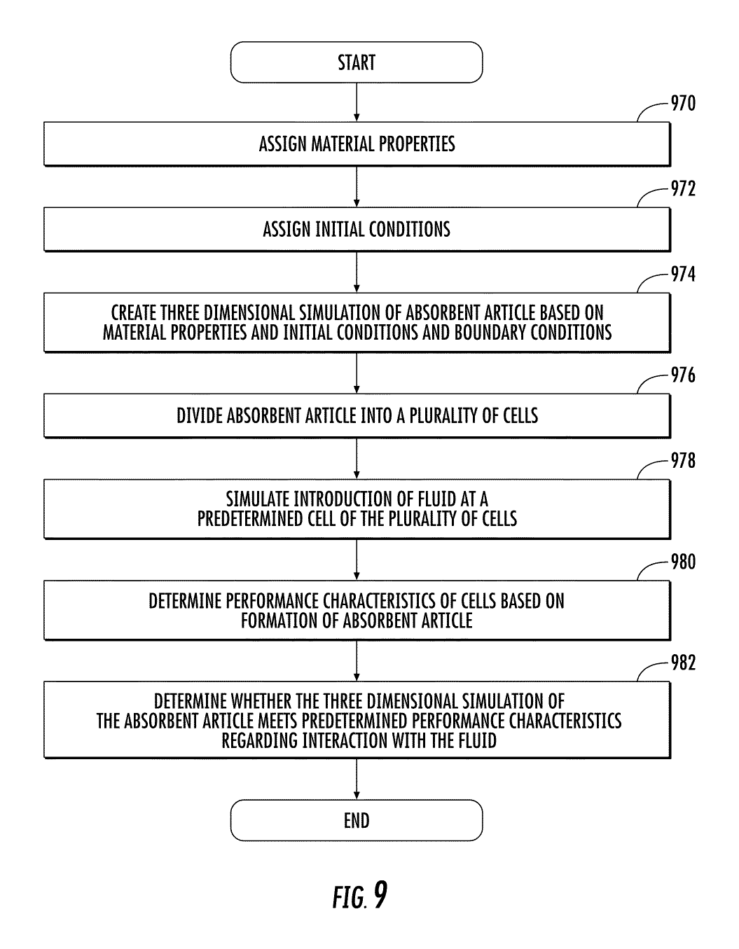

FIG. 9 depicts a flowchart for determining whether a simulated absorbent article meets predetermined performance characteristics, according to embodiments disclosed herein. As illustrated in block 970, one or more material properties may be assigned for the absorbent article. In block 972, one or more initial conditions may be assigned. In block 974, a three dimensional simulation of the absorbent article may be created based on material properties, the initial conditions, and the boundary conditions. In block 976 the three dimensional simulation of the absorbent article may be divided into a plurality of cells. In block 978, introduction of fluid at a predetermined portion or predetermined cell of the plurality of cells may be simulated. Depending on the particular embodiment, this simulation may be a simulation of the absorption of the absorbent article and/or removal of fluid from the absorbent article to a surface. For embodiments related to the removal of fluid from the absorbent article, the simulation may include simulating receipt of the fluid by the surface and thus may consider properties of the surface, as well as the absorbent article. Regardless, in block 980, performance characteristics of the cells may be determined based on formation and/or modeling of the absorbent article. In block 982, a determination may be made regarding whether the three dimensional simulation of the absorbent article meets predetermined performance characteristics regarding interaction with the liquid.

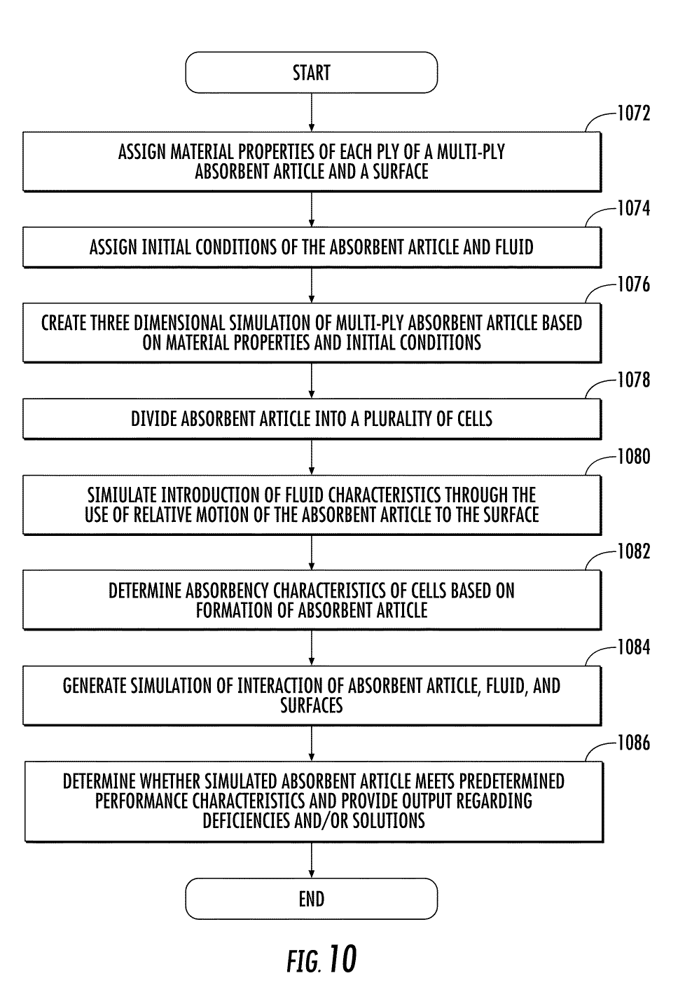

FIG. 10 depicts a flowchart for determining whether the simulated material meets predetermined performance characteristics, according to embodiments disclosed herein. As illustrated in block 1072, material properties may be assigned for each ply of a multi-ply absorbent article and a surface. In block 1074, one or more initial conditions and boundary conditions may be assigned for a fluid. In block 1076, a three dimensional simulation may be created for a multi-ply absorbent article, based on the material properties, the initial conditions, and the boundary conditions. It should be understood that while a three dimensional simulation may be created in this example, a two dimensional simulation, a three dimensional simulation, or greater than three dimensional simulation may also be created, depending on the particular embodiment.

In block 1078 the absorbent article may be divided into a plurality of cells. In block 1080, introduction of fluid characteristics may be simulated through use of relative motion of the absorbent article to the surface. In block 1082, absorbency characteristics of the cells may be determined, based on formation of the absorbent article. In bock 1084, simulation of interaction of the absorbent article, fluid, and surface may be simulated. In block 1086, a determination may be made regarding whether the simulated absorbent article meets predetermined performance characteristics. Output may be provided regarding deficiencies and/or solutions to the deficiencies. As an example, in response to a determination that the simulated absorbent article does not meet the predetermined performance characteristics, embodiments may be configured to determine a percent of fluid on an initial surface and determine saturation and/or a saturation distribution on the absorbent article.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be understood to those skilled in the art that various other changes and modifications can be made without departing from the spirit and scope of the invention. It is therefore intended to cover in the appended claims all such changes and modifications that are within the scope of this invention.

What is claimed is:

1. A system for modeling an absorbent article, comprising:
a memory component that stores logic that, when executed by a processor, causes the system to perform at least the following:
assign at least one material property of the absorbent article, wherein the absorbent article includes a plurality of absorbent plies, and wherein the absorbent article includes a plurality of absorbent regions;
assign at least one initial condition and at least one boundary condition associated with a fluid to be virtually introduced to the absorbent article;
perform a reduced order approximation to create a simulation of the plurality of absorbent regions, based on the at least one material property and the at least one initial condition; and
provide the simulation for display.

2. The system of claim 1, wherein the logic further causes the system to simulate an introduction of fluid characteristics of the fluid through use of relative motion of the simulation of the absorbent article with a surface.

3. The system of claim 1, wherein the logic further causes the system to determine a boundary condition, wherein the boundary condition identifies a source of the fluid and wherein the simulation utilizes the boundary condition.

4. The system of claim 1, wherein the logic further causes the system to simulate a surface on which the fluid resides.

5. The system of claim 1, wherein the reduced order approximation includes at least one of the following: a shell formulation.

6. The system of claim 1, wherein the reduced order approximation converts an n dimension initial condition into an n-1 dimension simulation.

7. The system of claim 1, wherein the simulation treats at least one ply of the plurality of absorbent plies as substantially flat from a microscopic level.

8. A method for modeling an absorbent article, comprising:
assigning, by a computing device, at least one material property of the absorbent article, wherein the absorbent article includes a plurality of absorbent plies;
assigning, by the computing device, at least one initial condition and at least one boundary condition associated with a fluid to be virtually introduced to the absorbent article;
performing, by the computing device, a reduced order approximation to create a simulation of the absorbent article, based on the at least one material property and the at least one initial condition; and
providing, by the computing device, the simulation for display.

9. The method of claim 8, further comprising simulating an introduction of fluid characteristics of the fluid through use of relative motion of the simulation of the absorbent article with a surface.

10. The method of claim 8, further comprising determining a boundary condition, wherein the boundary condition identifies a source of the fluid and wherein the simulation utilizes the boundary condition.

11. The method of claim 8, further comprising simulating a surface on which the fluid resides.

12. The method of claim 8, wherein the reduced order approximation includes at least one of the following: a shell formulation.

13. The method of claim 8, wherein the reduced order approximation converts an n dimension initial condition into an n-1 dimension simulation.

14. The method of claim 8, wherein the simulation treats at least one ply of the plurality of absorbent plies as substantially flat from a microscopic level.

15. A non-transitory computer-readable medium for modeling an absorbent article that stores logic that causes a computing device to perform the following:
assign at least one material property of the absorbent article, wherein the absorbent article includes a plurality of absorbent plies, wherein the absorbent article includes a plurality of absorbent regions;
assign at least one initial condition and at least one boundary condition associated with a fluid to be virtually introduced to the absorbent article;
perform a shell formulation to create a simulation of the plurality of absorbent regions, based on the at least one material property and the at least one initial condition; and
provide the simulation for display.

16. The non-transitory computer-readable medium of claim 15, wherein the logic further causes the computing device to simulate an introduction of fluid characteristics of the fluid through use of relative motion of the simulation of the absorbent article with a surface.

17. The non-transitory computer-readable medium of claim 15, wherein the at least one boundary condition identifies a source of the fluid and wherein the simulation utilizes the at least one boundary condition.

18. The non-transitory computer-readable medium of claim 15, wherein the logic further causes the computing device to simulate a surface on which the fluid resides.

19. The non-transitory computer-readable medium of claim 15, wherein the reduced order approximation converts an n dimension initial condition into an n-1 dimension simulation.

20. The non-transitory computer-readable medium of claim 15, wherein the simulation treats at least one ply of the plurality of absorbent plies as substantially flat from a microscopic level.
